(19) United States
(12) Patent Application Publication
(10) Pub. No.: US 2013/0064912 A1
(43) Pub. Date: Mar. 14, 2013

(54) FORMULATION FOR ALLEVIATION OF KIDNEY STONE AND GALLSTONE SYMPTOMS

(71) Applicant: Jon Barron, Chatsworth, CA (US)

(72) Inventor: Jon Barron, Chatsworth, CA (US)

(21) Appl. No.: 13/605,602

(22) Filed: Sep. 6, 2012

Related U.S. Application Data
(60) Provisional application No. 61/532,865, filed on Sep. 9, 2011.

(51) Int. Cl. A61K 36/185; A61K 36/56; A61K 36/28; A61K 36/14; A61K 36/899; A61K 36/45; A61K 36/23; A61K 36/73; A61K 36/288; A61K 36/752; A61K 36/534; A61P 13/12; A61P 13/00; A61P 13/02; A61P 31/00; A61P 13/08; A61K 36/29

(52) U.S. Cl. 424/747

(57) ABSTRACT

A nutraceutical formula and food-based medicinal composition is provided comprising homeopathic ingredients in combination with traditional herbal and nutraceutical ingredients for relieving the symptoms associated with presence or formation of kidney stones and gallstones. The formulation includes ingredients balanced in a synergistic manner to elicit complementary effects which both reduce the symptoms of kidney stones and gallstones and provide the potential to eliminate their occurrence in the body and prevent future recurrence. Methods of making and using core medicinal formulations and food-based compositions are also described.

This application claims the benefit of earlier filed U.S. Provisional Application No. 61/532,865, filed on Sep. 9, 2011, which is hereby incorporated by reference herein.

### FIELD OF THE INVENTION

A nutraceutical formula and a food-based homeopathic medicine including homeopathic ingredients in combination with traditional herbal and/or nutraceutical ingredients for relieving symptoms associated with kidney stones and/or gallstones is provided. The formulation includes ingredients balanced in a synergistic manner to elicit complementary effects which reduce the symptoms of kidney stones and/or gallstones in the body. The formulation also offers the potential to dissolve pre-existing stones and/or prevent the formation of new stones. Methods of making both the core medicinal formulation and suitable food-based homeopathic medicines are also described.

### BACKGROUND

Diseases of the kidneys range from mild infection to life-threatening kidney failure. The most common form of kidney disease is an inflammation of the kidneys caused by kidney sludge. Kidney sludge is the result of the accumulated crystallized minerals that sometimes obstruct the flow of urine and damage the kidneys. Kidney sludge may not be painful passing out the ureter, but it can nevertheless be deadly over time as it slowly chokes off kidney function nephron by nephron. If the minerals accumulate to a sufficient degree, the sludge can form rough surfaced stones that can rip and tear at the ureters as they are passed out of the kidneys.

Virtually every living person has some degree of sludge build up and some loss of kidney function over time; the only question is—how much? Does it reach the point where it causes painful kidney stones to form or the point where it chokes off a critical mass of kidney tissue, ultimately leading to kidney failure?

Although kidney stones and gallstones are not identical, the mechanisms involved in their formation are similar. Thus, it is believed that the same formulas used for eliminating kidney sludge and kidney stones can also help remove gallstones.

According to the National Kidney & Urologic Diseases Information Clearinghouse (NKUDIC), for the year 2000 it is estimated that 177,496 adults 20 years or older were admitted to the hospital with “calculus of kidney and ureters” as a primary diagnosis. A combined survey of physician office and hospital outpatient visits for the year 2000 reveals that there were approximately 2,000,000 visits by adults 20 years and older who were diagnosed with “calculus of kidney and ureters.” In the same year there were approximately 2,700,000 visits by adults 20 years and older with “urolithiasis” listed as diagnosis.

The NKUDIC also estimates that each year, nearly 100,000 Americans are newly diagnosed with kidney failure. More than 100,000 currently have End Stage Renal Disease (ESRD) due to diabetes, and an astounding 7.7 million have physiological evidence of chronic kidney disease (National Institute of Health (NIH), National Institute of Diabetes and Digestive and Kidney Diseases (NIDDK), “Kidney and Urological Diseases Statistics for the United States,” NIH Publication No. 11-3895, Aug 2011, available at http://kidney.niddk.nih.gov/KUDiseases/pubs/kustats/index.spx). According to the U.S. Health and Human Services Agency for Healthcare Research and Quality, an estimated 650,000 Americans will have kidney failure by 2010 and will require treatment via a renal replacement therapy, either ongoing renal dialysis or a kidney transplant. Without one of these therapies, ESRD is fatal.

Currently, there are no pharmaceutical drugs that can help with kidney stones. The only medical option is extracorporeal lithotripsy, which uses high-energy shock waves to fragment and disintegrate kidney stones. This is an expensive procedure at a cost of about $15,000 per treatment. Lithotripsy, however, can cause blood in the urine, is only effective for the treatment of large kidney stones, and can do little or nothing to eliminate kidney sludge.

Accordingly, there is a need and a desire for a therapeutic agent and/or treatment protocol which is effective for alleviating the symptoms associated with kidney stones and/or gallstones. There is an additional need and a desire for therapeutic agent and/or treatment protocol which can reduce the rate of or prevent the formation of calculi in the kidneys and/or gallbladder, such as by preventing or reducing the formation of sludge in these organs. There is a further need and a desire for a therapeutic agent and/or treatment protocol which is efficacious, cost effective, and readily available to individuals suffering or at risk for one or more of the symptoms and/or complications associated with the formation of kidney stones and/or gallstones. There is a still further need and desire for a non-surgical and/or non-pharmaceutical alternative to the current therapeutic and/or medicinal protocols for the treatment of urolithiasis and cholelithiasis.

Homeopathy is a form of alternative medicine in which practitioners treat patients using highly diluted preparations that causes the body to experience low level manifestations of the symptoms that it is trying to eliminate. In homeopathy, this is known as the “law of similars.” In a sense, it is not unlike the principle behind vaccination—where the vaccine triggers low level versions of the disease you are trying to prevent, thereby training the immune system to respond to the real disease state if and when it may arise. Because of their long standing use in the United States, the U.S. Congress passed a law in 1938 declaring that homeopathic remedies are to be regulated by the FDA in the same manner as nonprescription, over-the-counter (OTC) drugs. Thus, homeopathic remedies are required to meet certain legal standards for strength, quality, purity, and packaging.

Homeopathic ingredients can be used to treat symptoms related to kidney stones and gallstones. Other remedies are used to treat urinary tract infections. Still other homeopathic remedies function as diuretics and can help remove excess water build-up from body tissues. Such remedies may be obtained from a pharmacy that carries a range of homeopathic medicines.

Traditional medicine (also known as indigenous medicine) comprises medical knowledge systems that have developed over generations within various societies before the era of modern medicine. Practices known as traditional medicines include herbal, Ayurveda, Siddha medicine, Unani, ancient Iranian medicine, Islamic medicine, traditional Chinese medicine, traditional Korean medicine, acupuncture, Muti, Ifá, traditional African medicine, and other medical knowledge and practices all over the globe. These and other knowledge systems are actively being studied in branches of science such as ethnopharmacology and ethnobotany. Although traditional medicine has no legal standing in the United States, it is nevertheless global in application, particularly in the Far East, where it has been estimated that up to 80% of the population continues to use these traditional methods to treat primary medical problems. In the past decade or so, research has been increasingly focused on scientific evaluation of traditional medicines and drugs of plant and herbal origin, including methods derived from indigenous or tribal populations. Thus, traditional medicines can include many well-known herbs and spices.

If a way could be found to use herbal and nutraceutical ingredients in combination with homeopathic ingredients, balanced in a synergistic manner to elicit complementary effects which both reduce the symptoms associated with kidney stones and/or gallstones and provide the potential to dissolve such stones and/or prevent their formation, this would represent a useful contribution to medical and nutritional science.

### SUMMARY OF THE INVENTION

A general object of the invention is to provide a medicinal formulation which is effective for preventing and/or alleviating the symptoms associated with the formation of kidney stones and/or gallstones.

A more specific object of the invention is to overcome one or more of the problems described above, that is, diseases of the kidney such as nephrolithiasis (kidney stones), glomerulonephritis, and kidney infections or other urologic conditions such as ureter infections, bladder infections, or urinary tract infection (UTI).

In one embodiment, a liquid medicinal formulation for reducing or alleviating the symptom associated with kidney stones and/or gallstones contains one or more homeopathic ingredients such as Berberis vulgaris and Nux vomica, and one or more liquid pressings or extracts of traditional and/or nutraceutical ingredients. The traditional and/or nutraceutical ingredients can be selected from agrimony herb, chanca piedra herb, corn silk, dandelion leaf, goldenrod herb, gravel root, horsetail herb, hydrangea root, juniper berry, marshmallow root, orange peel, parsley root, peppermint leaf, uva ursi leaf, and combinations thereof.

In another embodiment, a liquid medicinal formulation can be in the form of a food-based homeopathic medicine which includes a homeopathic ingredient and a traditional and/or nutraceutical ingredient blended with a fruit and/or vegetable juice. The food-based homeopathic medicine can have a serving size of about 4 to about 32 fluid ounces.

Also provided is a method for reducing the symptoms associated with the formation of kidney stones and/or gallstones which includes administering to an individual in need thereof a liquid medicinal formulation including at least one homeopathic ingredient combined with a complementary traditional and/or nutraceutical ingredient. The liquid medicinal formulation can be food-based such as, for example, a ready-to-drink (RTD) beverage.

### DETAILED DESCRIPTION

One or more of the above objects can be achieved, at least in part, by utilizing a nutritional composition comprising a complementary combination of homeopathic and traditional medicine components or ingredients. Use of the components singularly generally does not produce the desired effect of relief from the symptoms of kidney stones and/or gallstones. However, it has been discovered that the advantageous effects of reducing and/or alleviating the symptoms associated with kidney stones and/or gallstones can be achieved by combining the ingredients in a multi-component formula. It is believed that utilizing a multi-component combination, in various embodiments as described herein, is important to provide the desired functionality. Single component remedies, including pharmaceutical drugs (i.e., monotherapy) have not been found to achieve this effect.

Compositions disclosed herein offer a multi-faceted approach to preventing, reducing and/or alleviating the symptoms associated with the formation of kidney stones and/or gallstones by providing an effective combination of ingredients that have anti-lithic (stone breaking), diuretic (water removing), antiseptic (infection killing), anti-nephrotoxic, anti-inflammatory, and/or anti-hepatotoxic properties. Additionally, such compositions are believed to be soothing to urinary tract tissue and stimulating or activating toward renal tissue.

In one aspect, a nutraceutical formulation or composition and/or a food-based medicine composition can include one or more homeopathic ingredients in combination with traditional herbal and nutraceutical ingredients to relieve the symptoms associated with the formation of kidney stones and/or gallstones. The homeopathic ingredients may also be efficacious as anti-pathogenic agents. Certain homeopathic ingredients as described herein have a body of research supporting their efficacy in: relieving pain associated with kidney stones and/or gallstones; and in relieving urinary discomfort and providing urinary function support. Embodiments of the formulation include ingredients balanced in a synergistic manner to elicit complementary effects which reduce the pain associated with kidney stones and/or gallstones, provide the potential to dissolve said stones, and reduce the likelihood of recurrence. It would also be desirable to reduce the size of the stones or break up the stones (by nutraceutical treatment alone, or concomitant with lithotripsy, i.e., pulverization or fragmentation) so that they can be more easily eliminated or discharged from the body. Methods of making both the core medicinal formulation and suitable food-based embodiments are described herein.

In one embodiment, a method for reducing symptoms associated with the formation or presence of kidney calculi or gallbladder calculi in a human patient may include administering to the patient in need thereof a medicinal composition as described in an amount effective to provide relief from at least one symptom associated with kidney or gallbladder calculi. The symptoms may include, but are not limited to urinary discomfort or pain in the patient, urinary dysfunction, abnormal urine odor, urinary leakage; urinary incontinence, urinary leakage, urinary hesitancy, weak urination, urinary blockage, urinary dribbling, nocturnal enuresis, urinary urgency, or increased urinary frequency. Further symptoms may include, but are not limited to, urinary tract infection (UTI), urethral infection, ureter infection, kidney infection, prostatitis, bladder infection, or gallbladder infection.

Possible homeopathic ingredients for embodiments of the invention can include, among others, the following.

Berberis vulgaris (Barberry) is used to help reduce the symptoms of cramping, inflammation, irritation, and discomfort associated with kidney and renal system dysfunction. It is also used to lessen the shooting pain associated with gallstones and kidney stones.

Nux vomica acts as a pain reliever in conditions associated with kidney stones, gallstones, and cystitis.

Calcarea carbonicum (a.k.a. Calcarea carbonica) is typically used when a patient complains of dark brown colored urine, resulting from bleeding caused by kidney stones. It is part of many homeopathic formulations designed to treat symptoms associated with kidney stones.

Traditional and/or nutraceutical ingredients for use in combination with the homeopathic component(s) are suitably selected to provide additional and/or complementary benefits and/or properties. For example, complementary and/or synergistic ingredients may be efficacious as anti-lithic (stone breaking) agents, diuretic (water removing) agents, antiseptic (infection killing) agents, anti-nephrotoxic (neutralizes kidney toxins) agents, and/or anti-hepatotoxic (blood cleansing) agents. Additionally or alternatively, such traditional and/or nutraceutical ingredients can be soothing to urinary tract tissue, have anti-inflammatory properties, and/or stimulate renal tissue. Juniper, for example, is known to be a stimulating diuretic and a great healer to the kidneys and urinary tract. (Dr. John Christopher, “School of Natural Healing,” (Springville, Utah: Christopher Publications, 1996), p. 273.)

Examples of traditional medicines that can be used in kidney stone and gallstone formulations include various herbal ingredients, or extracts thereof, such as, but not limited to the following ingredients. As used herein the term “alcohol” means ethyl alcohol (ethanol), unless otherwise specified. When alcohol is given in terms of % vol./vol. it is to be generally understood that the balance is water or aqueous liquid, unless stated otherwise.

As used herein, the word “tincture” refers to an initial, base liquid from which other preparations are derived, or which may be further diluted for use. Generally, a tincture is a concentrated extract of a natural material, namely, a botanical, herbal, plant, or animal substance. Such extracts are suitably prepared using aqueous or alcoholic solvents or mixtures thereof. Accordingly, a tincture may be prepared using a solvent selected from, but not limited to, ethyl alcohol, water, or mixtures thereof. Such extracts are susceptible to further dilution and are understood to be compatible with edible liquids.

As used herein, the phrase “mother tincture” (MT) refers to the initial base liquid from which homeopathic dilutions are made. The number of times the mother tincture is diluted with an equal amount of solvent is referenced by the number appearing before the X, as in 6×, 12×, and 30×.

Exemplary herbal and homeopathic remedies are described below.

Agrimony (Agrimonia eupatoria) is one of the most frequently used herbal supplements for kidney stones. Primarily because of its high silica content, Agrimony may reduce the duration of the symptoms associated with kidney and gallstones and may assist in the dissolution or discharge of kidney stones over a short period of time such as, for example, within a matter of weeks. Urinary incontinence, cystitis and other disorders of this system may also be treated with Agrimony.

Chanca piedra (Phyllanthus niruri) works equally well on gallstones, kidney stones, and kidney sludge. The name chanca piedra, as it is known in Peru, comes from its effect on kidney stones and gallstones. The literal translation is “stone breaker.” It effectively softens both kidney stones and gallstones for easy passage out of the body. It is also renowned for its diuretic qualities and has been shown effective at helping relieve edema and urine retention. It also works as an anti-inflammatory agent in the kidneys and as an antihepatotoxic in the liver. That is to say, it counters the effects of toxins in the liver. (See: Murugaiyah V., et al., “Antihyperuricemic lignans from the leaves of Phyllanthus niruri,” Planta Med. (2006) Nov, 72(14): 1262-7; Micali, S., et al., “Can Phyllanthus niruri affect the efficacy of extracorporeal shock wave lithotripsy for renal stones? A randomized, prospective, long-term study,” J. Urol. (2006) Sep, 176(3): 1020-2; Barros, M. E., et al., “Effect of extract of Phyllanthus niruri on crystal deposition in experimental urolithiasis,” Urol. Res. (2006) Aug, 34(6): 351-7; Nishiura, J. L., et al., “Phyllanthus niruri normalizes elevated urinary calcium levels in calcium stone forming (CSF) patients,” Urol. Res. (2004) Oct, 32(5): 362-6.; and Barros, M. E., et al., “Effects of an aqueous extract from Phyllanthus niruri on calcium oxalate crystallization in vitro,” Urol. Res. (2003) 30(6): 374-9.)

Corn silk (Maydis stigma) is a soothing diuretic and works as an excellent remedy for urinary conditions such as retained urine, burning urine, kidney stones, bladder infections, gonorrhea, and as a lymphatic system cleanser. Corn Silk is used to treat bladder infections, kidney stones, infections of the prostate gland, and urinary infections. (See, Maksimovic, Z., Dobric, S., Kovacevic, N., and Milovanovic, Z., “Diuretic activity of Maydis stigma extract in rats,” Pharmazie (2004) Dec; 59(12): 967-71.)

Dandelion leaves and roots (Taraxacum officinale) have been used for centuries to treat liver, gall bladder, kidney, and joint problems. Dandelion leaf is also a good natural source of potassium, and will replenish any potassium that may be lost due to the diuretic action of the other herbs in this formula. In addition, studies show beneficial effects of dandelion on reducing urinary tract gravel, attributed to disinfectant action and possibly the presence of saponins (See, Clare, B. A., Conroy, R. S., and Spelman, K., “The diuretic effect in human subjects of an extract of Taraxacum officinale folium over a single day,” J. Ahern. Complement Med. (2009) Aug, 15(8): 929-34; and Committee on Herbal Medicinal Products, “Community Herbal Monograph on Taraxacum Officinale, Weber ex Wigg., Radix Cum Herba,” European Medicines Agency, London, 2009.)

In one embodiment, Taraxacum officinale tincture may be prepared from the leaves of the plant, using from about 45% vol./vol. to about 65% vol./vol. alcohol. In another embodiment, Taraxacum officinale tincture may be prepared from the whole plant, optionally gathered before the development of flowers, using from about 45% vol./vol. to about 65% vol./vol. alcohol.

Goldenrod (Solidago virgaurea) is used as an aquaretic agent, meaning that it promotes the loss of water from the body (as compared to a diuretic, which promotes the loss of both water and electrolytes such as salt). It is used frequently in Europe to treat urinary tract inflammation and to prevent or treat kidney stones. Goldenrod has received official recognition in Germany for its effectiveness in getting rid of kidney stones, and it is commonly found in teas to help “flush out” kidney stones and stop inflammatory diseases of the urinary tract. Goldenrod is said to wash out bacteria and kidney stones by increasing the flow of urine, and also, soothe inflamed tissues and calm muscle spasms in the urinary tract. Several studies have found that goldenrod does in fact increase urine flow. (See, Melzig, M. F., “Goldenrod—a classical exponent in the urological phytotherapy,” Wien. Med. Wochenschr. (2004) Nov, 154(21-22): 523-7.)

In one embodiment, Solidago virgaurea tincture may be prepared from the whole plant, using from about 45% vol./vol. to about 65% vol./vol. alcohol. In another embodiment, Solidago virgaurea tincture may be prepared from the flowers (or, flowering tops), using from about 45% vol./vol. to about 65% vol./vol. alcohol.

Gravel root (Eupatorium purpureum). Like chanca piedra and hydrangea, gravel root also exhibits both diuretic and anti-lithic properties. Used primarily for kidney stones or gravel (which accounts for its name), it also helps with cystitis, dysuria, urethritis, and pelvic inflammatory disease. It can also play a role in the systemic treatment of rheumatism and gout as it promotes excretion of excess uric acid. And finally, it tones the reproductive tract and is used to treat inflammation of the prostate. (See, BHMA 1983 British Herbal Pharmacopoeia, BHMA, Bournemouth; and Grieve, M., A Modern Herbal: The Medicinal, Culinary, Cosmetic, and Economic Properties, Cultivation and Folk-Lore of Herbs, Grasses, Fungi, Shrubs, and Trees with All Their Modern Scientific Uses, Reprint of the 1931 ed., C.F. Leyel, Ed., London, 1985.)

In one embodiment, Eupatorium purpureum tincture may be prepared from the root of the plant.

Horsetail (Equisetum arvense) has not been extensively studied in people, however, professional herbalists recognize that the herb has diuretic (promotes the excretion of urine) properties that may be useful for the treatment of urinary tract infections and kidney stones (Lemus, I. et al., “Diuretic activity of an Equisetum bogotense tea (Platero herb): evaluation in healthy volunteers,” J. Ethnopharmacol. (1996) 54:55-8).

In one embodiment, Equisetum arvense tincture may be prepared from the stems of the plant, using from about 45% vol./vol. to about 65% vol./vol. alcohol.

The most common use for hydrangea (Hydrangea arborescens) is for the kidneys and bladder because of its effective diuretic property which helps increase the flow of urine. This removes impurities from the system and lessens the likelihood of infection along the entire urinary tract, which includes the kidneys, bladder, prostate (in men) and urethra. Hydrangea, like chanca piedra, is also considered an anti-lithic herb, which prevents stones or gravel from forming in the kidneys and bladder. As an anti-lithic herb, it can also assist the body in removing stones and gravel from these organs. This was a primary use of hydrangea by Native Americans. (See, Li, W. et al., “Total synthesis of adicardin,” J. Asian Nat. Prod. Res. (2009) Aug, 11(8): 720-7.)

In one embodiment, Hydrangea arborescens tincture may be prepared from the root of the plant, using from about 45% vol./vol. to about 65% vol./vol. alcohol. Alternatively, Hydrangea arborescens tincture may be prepared from the leaves and/or shoots of the plant, using from about 45% vol./vol. to about 65% vol./vol. alcohol.

Juniper berries (Juniperus communis) are used to treat infections, especially within the urinary tract, bladder, kidneys, and prostate. Their antiseptic properties help remove waste and acidic toxins from the body, stimulating a fighting action (or immune reaction) against bacterial and yeast infections. As a diuretic, Juniper Berries eliminate excess water retention contributing to weight loss. Juniper Berries make an excellent antiseptic in conditions such as cystitis. Contemporary herbalists primarily use juniper as a diuretic (“water pill”) component of herbal formulas designed to treat bladder infections. The volatile oils of juniper reportedly increase the rate of kidney filtration, thereby increasing urine flow and perhaps helping to “wash out” or otherwise eliminate offending bacteria. The volatile oils of juniper include isomers of terpineol, particularly terpinen-4-ol, which may cause an increase in urine volume. According to some sources, juniper increases urine volume without a loss of electrolytes such as potassium. (See, Blumenthal, M., Goldberg, A., and Brinckman, J., Herbal Medicine: Expanded Commission E Monographs, (Newton, Mass.: Integrative Medicine Communications, 2000), pp. 218-20.) It is recommended by the German Commission E for kidney ailments. (See, Yarnell, E., “Botanical medicines for the urinary tract,” World J. Urol. (2002) Nov, 20(5): 285-93, Epub 2002 Oct 17.)

In one embodiment, Juniperus communis tincture may be prepared from the ripe berries of the plant, using from about 45% vol./vol. to about 65% vol./vol. alcohol.

Marshmallow root's (Althaea officinalis) highest medicinal acclaim is as a demulcent. Internally, it has a soothing effect on inflamed and irritated tissues of the alimentary canal, and urinary and respiratory organs. It aids in the passage of kidney stones and is used in combination with other diuretic herbs for kidney treatments which assist in the release of gravel and stones. It works very well for urinary problems. (See, Guarnieri, A., Chiarini, A., Burnelli, S., and Amorosa, M., “Mucilage of Althaea officinalis,” Farmaco Prat. (1974) Feb, 29(2): 83-91.)

Orange peel (Citrus reticulata): Limonene and flavonoids found in orange peel may have anti-carcinogenic properties. They can block the carcinogenesis by acting as a blocking agent. Studies have shown that limonin and limonene can induce the enzyme activity of glutathione S-transferase, which is an important detoxifying enzyme. In addition, orange peel has antiseptic, bactericidal, and fungicidal properties. (See, Dayanand Dubey, K., Balamurugan, R. C., Agrawal, Rahul Verma, and Rahi, Jain, “Evaluation of Antibacterial and Antioxidant Activity of Methanolic and Hydromethanolic Extract of Sweet Orange Peels,” Recent Research in Science and Technology (2011) 3(11): 22-25.) Limonene as used herein includes d-limonene.

Parsley root (Petroselinum crispum) is an important diuretic that also helps clear uric acid from the urinary tract and helps dissolve and expel gallstones and gravel—and prevent their future formation. Parsley root is also known as Petroselinum hortense and Petroselinum sativum. A decoction of parsley root can help eliminate bloating and reduce weight by eliminating excess water gain. Note: the German Commission E, an advisory panel on herbal medicines, has approved parsley for use in the prevention and treatment of kidney stones. (See, Blumenthal, M., Busse, W. R., and Goldberg, A., et al. (eds), The Complete German Commission E Monographs, 1st ed., Austin, Tex.: American Botanical Council, 1998.)

In one embodiment, Petroselinum crispum tincture may be prepared from the whole plant. In another embodiment, Petroselinum crispum tincture may be prepared from the root of the plant.

Peppermint leaf (Mentha piperita) has a relaxing effect on the muscles of the digestive and urinary system. It is useful for treating spasm problems in the urinary tract. It also has strong antibacterial and anti-fungal properties which help rid the kidneys of bacteria. (See, Schelz, Z., Molnar, J., and Hohmann, J., “Antimicrobial and antiplasmid activities of essential oils,” Fitoterapia (2006) Jun, 77(4): 279-85, Epub 2006 May 11.)

In one embodiment, Mentha piperita tincture may be prepared from the whole plant, using from about 45% vol./vol. to about 65% vol./vol. alcohol. In another embodiment, Mentha piperita tincture may be prepared from the leaves of the plant, using from about 45% vol./vol. to about 65% vol./vol. alcohol.

One bioactive constituent of Uva Ursi (Arctosyaphylos uva ursi) is a glycoside called arbutin. This is what is responsible for its diuretic action. During its excretion arbutin produces an antiseptic effect on the urinary mucous membrane and can therefore help eliminate urinary tract infections. Tannic acid is also contained in the leaves. This herb also helps to keep the pH balance of urine from being too acidic. It may also strengthen the lining of the urinary tract and help to relieve any inflammation in the system. It has a direct sedative effect on the bladder walls. Allantoin, another bioactive component also found in Uva Ursi is believed to spur the healing of wounds. For chronic inflammation of the bladder or kidneys Uva Ursi has no equal. Two studies report that urine from individuals given Uva Ursi is active against the most commonly involved bacteria in bladder and urinary tract infection. This study supports the results of a double blind study of 57 women with recurrent cystitis. After one year, the placebo group had 20% incidence of recurring cystitis, whereas the group given Uva Ursi had no recurring infection. In addition it has anti-lithic properties that help in dissolving crystals not just in the kidneys, but throughout the body as well. It has, therefore, been used for arthritis and other painful joint problems. (See, Head, K. A., “Natural approaches to prevention and treatment of infections of the lower urinary tract,” Ahern. Med. Rev. (2008) Sep, 13(3):227-44; and Larsson, B., Jonasson, A., and Fianu, S., “Prophylactic effect of UVA-E in women with recurrent cystitis: A preliminary report,” Current Therapeutic Research (1993) 53(4): 441-43.)

In one embodiment, Uva Ursi (Arctosyaphylos uva ursi) tincture may be prepared from the leaves of the plant.

In addition to the homeopathic and traditional and/or nutraceutical ingredients described above, a nutritional or medicinal composition for the alleviation and/or prevention of the symptoms associated with kidney stones and gallstones can include one or more adjunct ingredients. Suitable adjunct ingredients for use in the formulations disclosed herein include, but are not limited to, extracts, tinctures, and/or powdered materials derived from one or more plant parts or extracts of such plant parts. Such adjunct ingredients may be selected from buchu, barberry, Fo Ti, Schizandra chinesis, fenugreek, Calcarea carbonicum, Lysmachia, and combinations thereof.

Buchu (Agathosma betulina) has been used by the natives of the Western Cape of Southern Africa for many centuries. Buchu has been used traditionally to help cleanse the urinary tract and act as a natural diuretic, helping to flush out and cleanse the bladder and entire urinary tract system.

As discussed above, Barberry (Berberis vulgaris) supports the urinary system, bladder and kidney function and can help alleviated such symptoms as cramping, inflammation, irritation and discomfort. It is excellent for kidney stones and gallbladder problems. Barberry may optionally be used as a homeopathic component or an adjunct component.

Fo Ti (Polygonum multiflorum) is a traditional tonic which can be used to support kidney and liver functions.

Schizandra chinensis is used by homeopathy practitioners and Chinese herbalists as a kidney tonic. It can used to relieve irritation, burning, and frequent urination.

Fenugreek (Trigonella foenum-graecum) is one of the oldest cultivated plants and the seed extract is used by traditional Chinese herbalists to treat or ameliorate the symptoms related to kidney problems. According to many herbalists, kidney problems can be effectively treated, prevented, and/or cured by using fenugreek.

Lysmachia, Jin Qian Cao, or “Gold Coin Herb” is often used for gallstones and for kidney stones.

As discussed above, Calcarea carbonicum is used to treat several symptoms associated with kidney stones. Calcarea carbonicum (a.k.a. Calcarea carbonica) may optionally be used as a homeopathic component or an adjunct component.

The nutraceutical compositions of the present invention may be administered in combination with a nutraceutically acceptable carrier. The active ingredients in such formulations may comprise from 1% by weight to 99% by weight, or alternatively, 0.1% by weight to 99.9% by weight. “Nutraceutically acceptable carrier” means any carrier, diluent or excipient that is compatible with the other ingredients of the formulation and not deleterious to the user. In accordance with one embodiment, suitable nutraceutically acceptable carriers can include ethanol, aqueous ethanol mixtures, vegetable glycerine, water, fruit and/or vegetable juices, and combinations thereof.

### Delivery System

Suitable methods of administration include, but are not limited to, sublingual, buccal, oral, and the like.

Suitable dosage forms include tablets, capsules, lozenges, solutions, suspensions, powders, gums, gels, chews, gummies, and confectionaries. Other sublingual delivery systems include, but are not limited to, dissolvable tabs under and on the tongue, liquid drops, and beverages. Edible films, hydrophilic polymers, oral dissolvable films or oral dissolvable strips can be used.

Liquid-based nutritional or dietary supplement compositions for oral administration can be prepared in water, juices, or other aqueous vehicles. Useful edible liquids may be selected from a fruit juice, a vegetable juice, apple cider vinegar, water, and combinations thereof. Useful liquid forms include solutions, suspensions, emulsions, and the like. Microemulsions and microencapsulations are contemplated. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone (PVP), polyvinyl alcohol (PVA), and the like. The liquid nutritional or dietary supplement compositions can be in the form of a solution, emulsion, syrup, gel, or elixir including or containing, together with the above enumerated ingredients or compounds, wetting agents, surfactants, dispersants, emulsifiers, sweeteners, coloring agents, and flavoring agents. Various liquid and powder nutritional compositions can be prepared by conventional methods.

The nutritional and dietary supplement compositions may or may not be presented in unit dosage forms and/or servings, depending on the delivery system and/or the end user. Unit dosage, for example, would be applicable to a ready-to-drink (RTD) delivery system. In such form, the preparation is subdivided into unit doses containing appropriate quantities of the nutritional or active component(s). The unit dosage form can be a packaged preparation, the package containing discrete quantities of preparation.

Solid nutritional compositions for oral administration in connection with a method for preventing or treating kidney stones and gallstones may optionally contain, in addition to the above enumerated nutritional composition ingredients or compounds: carrier materials such as corn starch, gelatin, acacia, microcrystalline cellulose, kaolin, dicalcium phosphate, calcium carbonate, sodium chloride, alginic acid, and the like; disintegrators including, microcrystalline cellulose, alginic acid, and the like; binders including acacia, methylcellulose, sodium carboxymethylcellulose, polyvinylpyrrolidone, hydroxypropyl methylcellulose, ethyl cellulose, and the like; and lubricants such as magnesium stearates, stearic acid, silicone fluid, talc, waxes, oils, colloidal silica, and the like. For example, the active agent may be combined with at least one excipient such as fillers, binders, humectants, disintegrating agents, solution retarders, absorption accelerators, wetting agents, surfactants, dispersants, emulsifiers, absorbents, or lubricating agents. Other useful excipients include calcium stearate, mannitol, xylitol, sweeteners, modified starch, carboxymethylcellulose, silica, gelatin, silicon dioxide, and the like. The usefulness of such excipients is well known in the art.

In one embodiment, the nutritional composition may be in the form of a liquid. In accordance with this embodiment, a method of making a liquid composition is provided.

Liquid nutritional compositions for oral administration in connection with a method for preventing or treating kidney stones and gallstones can be prepared in water or other aqueous vehicles. In addition to the above enumerated ingredients or compounds, liquid nutritional compositions can include suspending agents such as, for example, methylcellulose, alginates, tragacanth, pectin, kelgin, carrageenan, acacia, polyvinylpyrrolidone, polyvinyl alcohol, and the like. The liquid nutritional compositions can be in the form of a solution, emulsion, syrup, gel, or elixir including or containing, together with the above enumerated ingredients or compounds, wetting agents, sweeteners, coloring and/or flavoring agents. Various liquid and powder nutritional compositions can be prepared by conventional methods. Various ready-to-drink (RTD) formulations are contemplated.

The embodiments described herein can be assembled from liquids obtained through several separate or sequential processes, including, but not limited to, homeopathic processes, including but not limited to, steeping, tea-making, preparing infusions, succussing, and extracting.

Treatments or preparations of the herbal ingredients listed above may be carried out by several processes such as, but not limited to, extraction, infusion, heating, distillation, steeping, sonication, succussion, dilution, mixing, filtration, pressing, cold pressing, grinding, pureeing, pounding, pulverizing, chopping, and the like.

Homeopathic remedies are made from the elements found in nature—mineral, animal, and plant extracts are the base of these natural remedies which are then diluted through altering the degree of concentration to avoid creating side effects that can be disagreeable. Paradoxically, it is postulated that the more a homeopathic remedy is diluted, the more effectively a remedy will work.

There is a very distinct process for making homeopathic remedies. For example, when making a homeopathic remedy which is of plant or animal nature the extract, which generally includes a soluble substance or substances (or, alternatively, is admixed with another soluble substance), is dissolved in a mixture of alcohol (ethanol) and water (approximately ninety percent pure alcohol and ten percent distilled water (although the ratio can vary). This mixture is optionally set aside for two to four weeks. It is periodically shaken and then is press strained once it has cured. This formulation procedure yields a remedy referred to as a “mother tincture” (as discussed above). See the references that follow, for example, regarding preparation of additional specific mother tinctures.

As used herein the terms “homeopathic” and “homeopathic ingredient” are defined in accordance with The Federal Food, Drug and Cosmetic Act (21 U.S.C. 351), which is incorporated herein by reference, and includes those agents or drugs used in the practice of homeopathy and listed in the Homeopathic Pharmacopeia of the United States (HPUS), the entirety of which is also incorporated herein by reference. For example, various mother tinctures may be described in HPUS including ratios of components.

Also incorporated herein by reference are the following homeopathic texts: Materia Medica with Repertory, 9^{th} Edition, by William Boericke, M.D. (Santa Rosa, Calif.: Boericke & Tafel, 1927), and A Dictionary of Practical Materia Medica, reprinted 2006, by John Henry Clarke, M.D. (New Delhi: B. Jain Publishers, 2006).

The mother tincture referred to above is diluted to produce different remedy potencies. To do this one of two scales is usually employed: the decimal (x or “X”) and the centesimal (c or “C”). The decimal scale dilution factor (X) is 1:10 and the centesimal (C) is 1:100. Optionally, an alcohol/water mixture is used for dilution in various stages.

Between each of the stages of dilution, the diluted tincture is optionally succussed (shaken vigorously).

Berberis vulgaris is available in a wide variety of dilutions from Hahnemann Laboratories, Inc. (San Rafael, Calif.).

Nux vomica is available in a wide variety of dilutions from Hahnemann Laboratories, Inc. (San Rafael, Calif.).

Calcarea carbonicum (a.k.a. Calcarea carbonica) is available in a wide variety of dilutions from Hahnemann Laboratories, Inc. (San Rafael, Calif.).

For example, to produce a 1C potency of the Berberis vulgaris homeopathic ingredient, one drop of the mother tincture is added to 99 drops of an alcohol/water mixture and succussed (i.e., shaking by forcefully striking the container on an elastic surface). To produce a 2C potency, one drop of the 1C mixture is added to 99 drops of an alcohol/water mixture and succussed. The number of a homeopathic remedy shows how many times it has been diluted and succussed, for example, Berberis 6C has been diluted and succussed six times. Standard dilutions include 6×, 12×, and 30×.

Without being bound by theory, it is noted that statistical and probability considerations may apply in a given solute/solution combination where dilutions reach or exceed 24×( 1/10²⁴ dilution), in comparison with Avogadro's number. The embodiments as described are not intended to be limited merely by theoretical calculations, particularly where a statistical probability may apply.

Herbal extracts can be made by grinding up the herbs into a fine powder and then suspending into a solution of alcohol and water (e.g., alcohol vol. can range from about 45-65% of the total volume) until the powder is completely covered. The solution is subsequently regularly agitated or pulverized (e.g., by ultrasonication in the laboratory) over time and then pressed through a filtering medium to extract the bio-active ingredients. Dried herbs for making extracts are available from Pacific Botanicals, Grants Pass, Oregon and Starwest Botanicals in Rancho Cordova, Calif. The described method is one way of producing a mother tincture, for example.

Berberis vulgaris and Nux vomica preparations can be manufactured as described above in a variety of dilutions. Berberis vulgaris mother tincture may be prepared from the bark of the root. Nux vomica mother tincture may be prepared from the seeds, in coarsely powdered form (for example). Useful homeopathic dilutions may range from respective mother tinctures to about 1× dilution, and up to about 30× dilution.

In one embodiment, Berberis vulgaris may be used in a dilution range of from about 1× to about 30×. In another suitable embodiment, Berberis vulgaris may be used in a dilution range of from about 1× to about 12×. In yet another embodiment, Berberis vulgaris may be used in a dilution range of from about 1× to about 6×.

In one embodiment, Nux vomica may be used in a dilution range of from about 1× to about 30×. In another suitable embodiment, Nux vomica may be used in a dilution range of from about 1× to about 12×. In yet another embodiment, Nux vomica may be used in a dilution range of from about 1× to about 6×. In yet another embodiment, Nux vomica may be used in a dilution range of from about 1× to about 3×.

In another embodiment, Calcarea carbonica may be used in a dilution range of from about 1× to about 30×. The solid (calcium carbonate according to Hahnemann) may be prepared by trituration of oyster shell, which has been reduced to a fine powder.

In accordance with certain embodiments, a food-based homeopathic medicine for use in the treatment, prevention, and/or alleviation of the symptoms associated with kidney stones and/or gallstones can include a core medicinal or nutraceutical composition combined with a food or beverage component. In accordance with certain embodiments, a food-based homeopathic medicine for use in the treatment, prevention, and/or alleviation of the symptoms associated with kidney stones and/or gallstones can include a core medicinal or nutraceutical composition combined with a food matrix component. Thus, the medicinal composition may be administered in the form of a functional food. The core medicinal or nutraceutical composition can include one or more homeopathic agents, one or more traditional and/or nutraceutical agents, and, optionally, one or more adjunct agents, each selected to elicit complementary effects which may reduce the pain associated with kidney stones and/or gallstones, provide the potential to dissolve said stones, and reduce the likelihood of recurrence. The food or beverage component can include any food matrix or nutritional formulation with which the core medicinal or nutraceutical composition may be combined without detracting from the palatability and/or nutritional profile of the food or beverage component and/or without limiting the efficacy of the core medicinal or nutraceutical formulation. Examples of suitable food or beverage components include, but are not limited to, fruit juices, vegetable juices, and combinations thereof. In certain embodiments, a food-based medicine containing a core medicinal or nutraceutical composition as disclosed herein can be provided as a ready-to-drink beverage having a serving size of about 4 to about 32 fluid ounces.

In a further embodiment, the core medicinal or nutraceutical compositions and/or food-based medicine disclosed herein can be utilized in a method for treating or reducing the symptoms associated with the formation of kidney stones and/or gallstones. Such method can include administering to a person in need thereof an effective amount of a medicinal, nutraceutical, or food-based composition effective to reduce the severity of a symptom associated with the presence of kidney or gallbladder calculi, promote the dissolution and/or discharge of kidney or gallbladder calculi, and/or reduce the rate of formation of such calculi.

The nutritional compositions and methods described above may be further understood in connection with the following Examples.

### EXAMPLE 1

A core liquid medicinal or nutraceutical formulation in accordance with the disclosure herein includes about 10% by volume homeopathic components and about 90% by volume traditional herbal and nutraceutical components. The homeopathic components include Berberis vulgaris 6× and Nux vomica 6× in a 1:1 volume/volume ratio. The traditional herbal and nutraceutical components and relative concentrations are as disclosed in Table 1, below.

TABLE 1

| | Volume |
|---|---|
| Ingredient | (fl. oz.) |
| Chanca piedra extract | 33 |
| Gravel root extract | 13 |
| Hydrangea root extract | 13 |
| Marshmallow root extract | 7 |
| Juniper berry extract | 3.4 |
| Corn silk extract | 3.4 |
| Uva ursi extract | 3.4 |
| Parsley root extract | 3.4 |
| Agrimony extract | 3.4 |
| Dandelion leaf extract | 3.4 |
| Horsetail extract | 3.4 |
| Orange peel extract | 3.4 |
| Peppermint extract | 3.4 |
| Goldenrod extract | 3.4 |
| Total herbal mixture | 100 |

Note:
the alcohol content of the extracts can range from 45-55% of the total volume

Based on the amount of herbs used in the above formula an “herb strength ratio” is determined. For Example 1 that ratio is about 1:2.5. The herbs are used in the ratios provided in the table, prepared using 45% to 55% aq. ethanol vol./vol. A total of 8 lbs of herbs are used, and then extracted using 20 lbs (or about 2.5 gallons) of solvent. After pressing, about 2.5 gallons of extract liquid (i.e., tincture) is obtained containing the bioactive components extracted from 8 lbs of herbals. The homeopathic components are then subsequently added.

The final core formulation has a density of about 1 g/ml, and may include a serving size of approx. 7.87 g. The “herb weight equivalence” per serving is about 3170 mg.

### EXAMPLE 1A

A medicinal or nutraceutical formulation is prepared in accordance with Example 1 including d-limonene added in the amount of 3 mL per gallon of tincture.

### EXAMPLE 2

A food-based homeopathic medicine composition in accordance with the embodiments disclosed herein can be prepared by combining a 1 fl. oz. serving of the core medicinal or nutraceutical composition as described in Example 1 with a 15 fl. oz. of a 50/50 apple juice/water mixture. The food-based composition can be portioned and packaged in a 16 fl. oz. container as a ready-to-drink beverage.

### EXAMPLE 3

In accordance with an embodiment, a human individual afflicted with one or more kidney stones and/or gallstones is administered orally a once daily serving of a composition as described in Example 2. After about 1 day to about 14 days, it is expected that the symptoms associated with the presence of kidney stones and/or gallstones will be reduced in the individual.

### EXAMPLE 3A

In accordance with EXAMPLE 3, it is expected that after about 1 to 3 days, the rate of formation (or incidence) of kidney stones and/or gallstones (i.e., calculi) will be substantially reduced, in comparison to the date of initial treatment. At that point, readministration about once every 1-3 months should be sufficient to keep symptoms from reoccurring.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

I claim:

1. A medicinal composition comprising:
at least one homeopathic component selected from the group consisting of Berberis vulgaris, Nux vomica, Calcarea carbonicum, and combinations thereof;
at least one medicinal or nutraceutical component selected from the group consisting of chanca piedra, gravel root, hydrangea root, marshmallow root, juniper berry, corn silk, uva ursi, parsley root, agrimony, dandelion leaf, horsetail, orange peel, peppermint, goldenrod, and combinations thereof; and
optionally, a nutraceutically acceptable carrier.

2. The medicinal composition according to claim 1, wherein the nutraceutically acceptable carrier comprises water, ethanol, or a combination thereof.

3. The medicinal composition according to claim 1, further comprising an adjunct ingredient selected from the group consisting of buchu, barberry, Fo Ti, Schizandra chinesis, fenugreek, Lysmachia, and combinations thereof.

4. The medicinal composition according to claim 1, wherein the at least one homeopathic component is a combination of Berberis vulgaris present in a 6× dilution and Nux vomica present in a 6× dilution and in a 1:1 volume/volume ratio.

5. The medicinal composition according to claim 1, wherein the at least one homeopathic component comprises about 10% of the total volume of the composition.

6. A nutritional composition, comprising:
a medicinal composition according to claim 1; and
at least one food or beverage component.

7. The nutritional composition according to claim 6, wherein the at least one food or beverage component comprises an edible liquid.

8. The nutritional composition according to claim 6, wherein the composition comprises a ready-to-drink (RTD) formulation.

9. The nutritional composition according to claim 7, wherein the edible liquid is selected from the group consisting of a fruit juice, a vegetable juice, apple cider vinegar, water, and combinations thereof.

10. A method for reducing symptoms associated with the formation or presence of kidney calculi or gallbladder calculi in a human patient, comprising:
administering to the patient in need thereof a medicinal composition according to claim 1 in an amount effective to provide relief from at least one symptom associated with kidney or gallbladder calculi.

11. The method according to claim 10, wherein the medicinal composition is administered in the form of a functional food composition.

12. The method according to claim 10, wherein the medicinal composition is administered in an amount effective to promote the reduction, dissolution, fragmentation, elimination, discharge or a combination thereof of kidney or gallbladder calculi.

13. The method according to claim 10, wherein the medicinal composition is administered in an amount effective to reduce the rate of formation of calculi in the kidney or gallbladder.

14. The method according to claim 10, wherein the at least one symptom is urinary discomfort or pain in the patient.

15. The method according to claim 10, wherein the at least one symptom is selected from the group consisting of urinary dysfunction, abnormal urine odor, urinary leakage; urinary incontinence, urinary leakage, urinary hesitancy, weak urination, urinary blockage, urinary dribbling, nocturnal enuresis, urinary urgency, and increased urinary frequency.

16. The method according to claim 10, wherein the at least one symptom is an infection selected from the group consisting of urinary tract infection (UTI), urethral infection, ureter infection, kidney infection, prostatitis, bladder infection, and gallbladder infection.
